(19) United States
(12) Patent Application Publication
(10) Pub. No.: US 2016/0135939 A1
(43) Pub. Date: May 19, 2016

(54) MEDICAL TEXTILE AND METHODS OF MAKING THE SAME

(71) Applicant: ATEX TECHNOLOGIES, INC., Pinebluff, NC (US)

(72) Inventors: Sarah Beyer, Pinebluff, NC (US); John Druga, Pinebluff, NC (US)

(21) Appl. No.: 14/652,822

(22) PCT Filed: Dec. 17, 2013
(86) PCT No.: PCT/US13/75635

§ 371 Date: Jun. 17, 2015

Related U.S. Application Data
(60) Provisional application No. 61/738,121, filed on Dec. 17, 2012.

(51) Int. Cl. A61F 2/00; D03D 1/00; D04B 1/22

(52) U.S. Cl. CPC A61F 2/0063; D04B 1/22; D03D 1/00; A61F 2250/0015; A61F 2250/0097

(57) ABSTRACT

The preferred embodiments herein describe a medical textile having a first segment having a first density, a second segment having a second density, and a third segment having a third density. The first, second and third segments converge and whereby the first, second and third densities are substantially the same. Methods of making the article are also described.

### FIELD

The embodiments of the present invention are directed to textile articles for medical use and methods of making such articles.

### BACKGROUND

Textiles are used to in a number of different medical applications including wound care, surgical implants, and the like. In surgical applications, textile products are made for various implant procedures including vascular and cardiac applications as well as soft tissue repair, tendon and ligament repair, and the like.

In medical textiles, it is desirable in some situations to have an article made as a single unit. These singularly made articles eliminate added fabrication time and cost, but also minimize any chance of failure if the article is a single, cohesive unit rather than the joinder of parts. Whether the article is used in wound care, as an implant or other application, if the article cannot unravel or separate, it prevents discomfort or injury to the patient. If the article is implanted, any unraveling or separation while inside a patient could have serious medical consequences.

### BRIEF DESCRIPTION OF THE FIGURES

FIG. 1 is a perspective view of a first embodiment described herein.

FIG. 2A is a right side view of the embodiment shown in FIG. 1.

FIG. 2B is a left side view of FIG. 1 of the embodiment shown in FIG. 1.

FIG. 3A is a diagrammatic representation of a knitting methodology described herein.

FIG. 3B is a diagrammatic representation of a first knitting alternative methodology described herein.

FIG. 4 is a diagrammatic representation of a second knitting alternative methodology described herein.

FIG. 5 is a diagrammatic representation of a first alternative embodiment using the methodology described herein.

FIG. 6 is a diagrammatic representation of a second alternative embodiment using a combination of the methodologies described herein.

FIG. 7 is a diagrammatic representation of a weaving methodology described herein.

FIG. 8 is a diagrammatic representation of a first alternative weaving methodology.

FIG. 9 is a diagrammatic representation of a third alternative embodiment using the described weaving methodology described herein.

FIG. 10 is a diagrammatic representation of examples of further alternative embodiments using the methodologies described herein.

### DETAILED DESCRIPTION

The following description and accompanying figures demonstrate different configurations that an implantable article of manufacture may take to reflect the various aspects and features of the embodiments described herein. The illustrations and descriptions are not intended to limit the described attributes and features of the embodiments. Unless otherwise defined, all scientific and technical terms used herein are intended to have the same meaning as commonly understood by one of ordinary skill in the art.

As used herein the singular foil is include the plural. Positional terms such as upper, lower, top, bottom, anterior, posterior, and the like are used to describe the embodiments herein. It will be appreciated that the scope and spirit of the embodiments herein described are not limited to the selected forms. Moreover, it is noted that the figures provided are not drawn to any particular proportion or scale and that numerous variations may be made to the illustrated embodiments. Those of skill in the art will recognize that the described aspects and features of the embodiments are not limited to any particular form, and that one or more embodiments may be designed to include one or more of the features of the embodiments for use in a number of different applications.

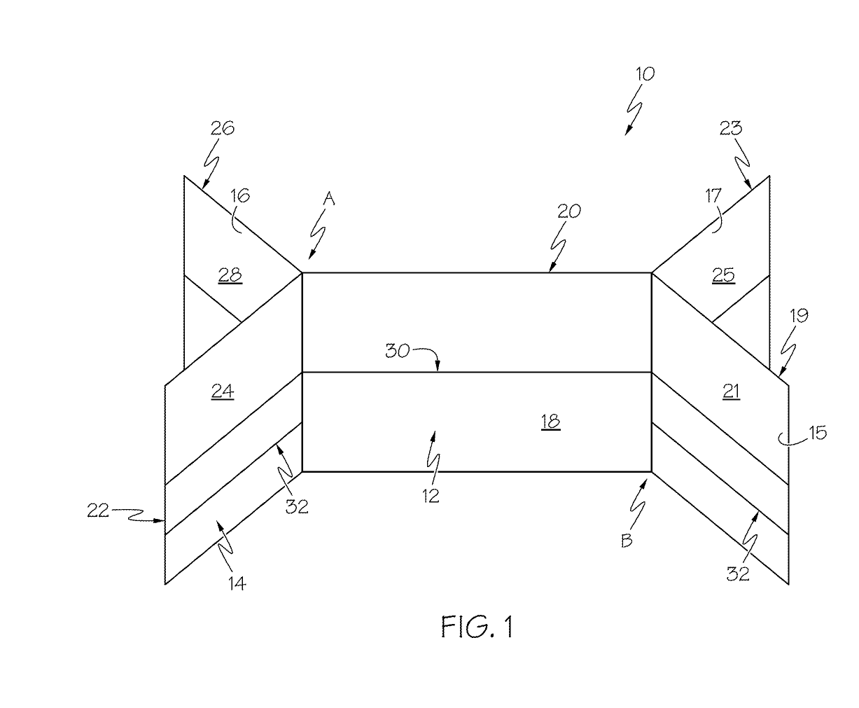

Turning now to FIG. 1, the first embodiment shows a medical article 10 having a first segment 12, formed from converging second 14 and third 16 segments at point A and from converging fourth 15 and fifth 17 segments at point B. The first segment 12 has a front surface 18 and a rear opposing surface 20. The second segment 14 has a second front surface 22 and a second rear opposing surface 24. Similarly, the third segment 16 has a third front surface 26 and a third rear opposing surface 28. The second front surface 22 and the third rear surface face 28 each other. The first segment 12 diverges at point B to form fourth 15 and fifth 17 segments. Fourth segment 15 has a fourth front surface 19 and a fourth rear opposing surface 21. Similarly, fifth segment 17 has a fifth front surface 23 and an opposing fifth rear surface 25. The fourth front surface 19 and the fifth rear surface 25 face each other.

One use for the articles described herein may be in a minimally invasive surgical technique. As a result, the article is fed by means of a catheter or the like, along a long and narrow channel to the site of the surgery. With such a delivery, it is difficult to ensure that the article is properly positioned before suturing to the patient. In a minimally invasive procedure, the surgeon has a limited visual range. This limited field of vision often does not provide the surgeon with a complete appreciation of the orientation of the article. It often happens that the article becomes skewed during travel through the delivery catheter and then becomes incorrectly transposed at the surgical site. When this happens, a surgeon may suture one segment of an article to the patient and then realize that the orientation of the article is wrong. This results in the surgeon having to cut those sutures, reorient the article to its proper position and resuture. This results in increased surgical time, expense, as well as possible complications to the patient. Thus, there is a need for an implanted medical article having relative positioning guides to aid a surgeon in a minimally invasive procedure.

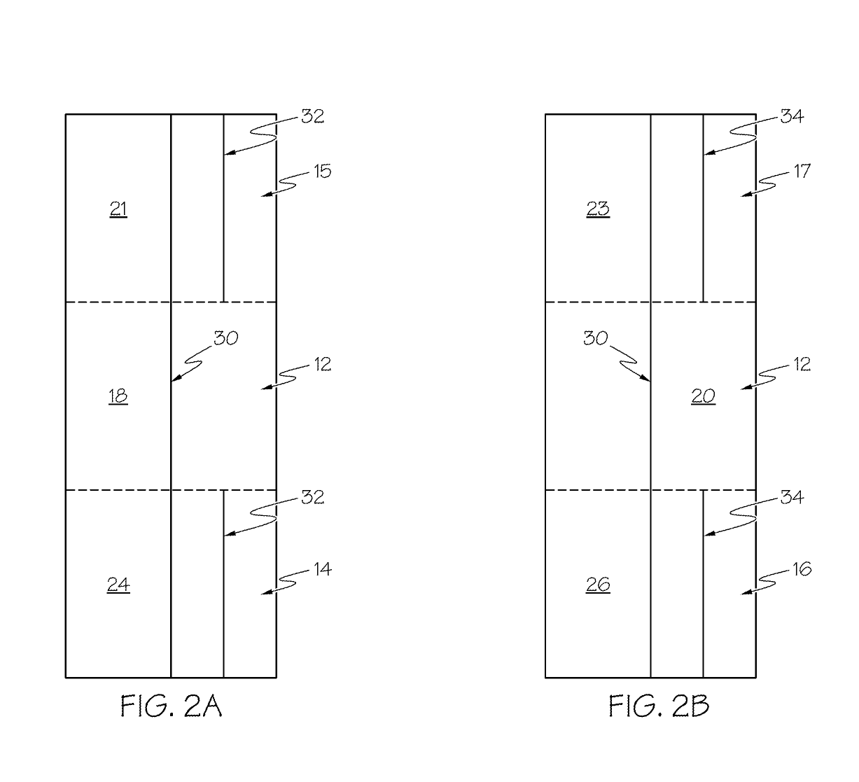

A central linear demarcation 30 is formed into the first 12, second 14, third 16, fourth 15, and fifth 17 segments. This aids the surgeon in accurately aligning the article 10 so that is it centrally located at the surgical site. Additional lines of demarcation are also formed into the article 10 to aid the surgeon in ensuring that the article is positioned accurately relative to all of its parts. For example, the second rear surface 24 of the second segment 14 and the fourth rear surface 21 of the fourth segment 15 have second outer demarcation 32 in the form of a line to the right of the central linear demarcation 30 as shown in FIG. 2A. Similarly the third front surface 26 of the third segment 16 and the fifth front surface 23 of the fifth segment 17 also have third outer demarcation 34 in the form of a line to the right of the central linear demarcation 30, as shown in FIG. 2B. This demarcation system provides a surgeon with a visual guide for the correct relative orientation of the article 10. For example, if the embodiment 10 becomes skewed during delivery via the catheter, and the second segment 14 front surface 22 is mistaken for the second segment rear surface 24, the surgeon will not view the second outer demarcation 32 to the right of the center demarcation 30 through the scope. The absence of this demarcation will inform the surgeon of the misalignment and result in the repositioning of the article 10 into its proper orientation prior to suturing. It should be appreciated that the second front surface 22 and the third rear surface face 28, and the fourth front surface 19 and the fifth rear surface 25 may have indicia to aid in relative positioning of the article 10 instead of or in addition to the indicia described above.

It should be understood that while the demarcation described above has included linear forms of demarcation, other forms such as the incorporation of color, pattern variations may also be used to provide a visual guide to ensure proper special orientation of the article prior to suturing or other permanent fixation into the patient.

The first 12, second 14, third 16, fourth 15 and fifth 17 segments are formed cohesively together as a single article. They may be knit or woven. Assuming that the lengths of each segment are relatively equal, the first segment 12 has a weight substantially the same as either the second 14, third 16, fourth 15 or fifth 17 segments. Substantially the same in this context means no more than 50% more. Because the segments are formed together as a single unit, different variables may be altered to obtain the similarity in weight between the segments. These include alteration of the stitch or weave pattern, pore size, variation in the courses per inch, wales per inch, picks per inch and ends per inch, and the incorporation of different yarns or filaments in different areas Each of these and the methodology adopted to create the article 10 will now be described in detail below.

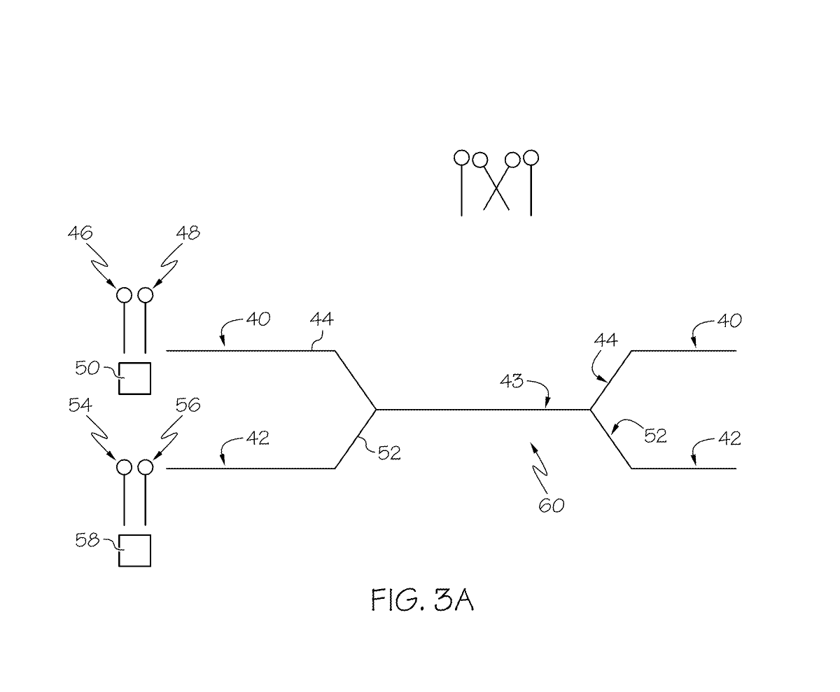

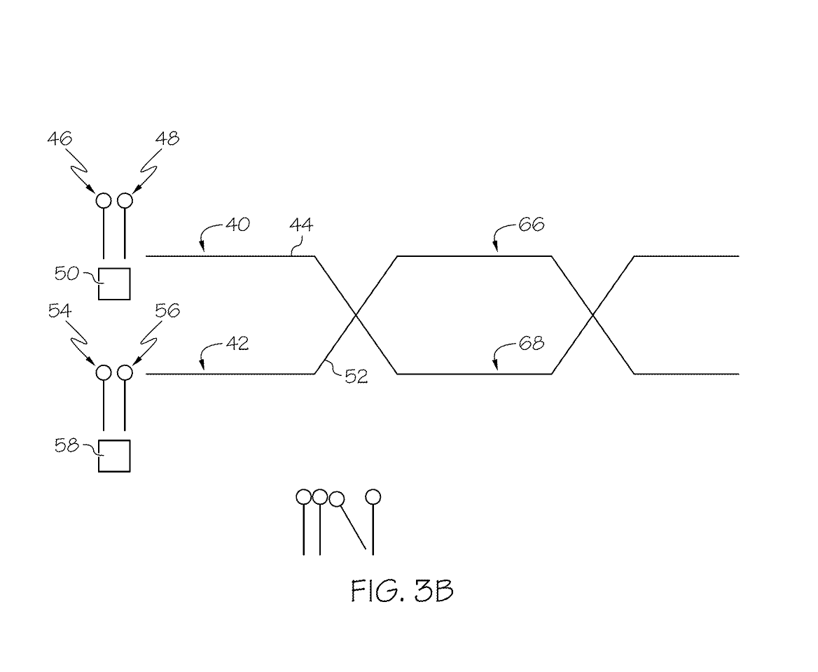

The textile article 10 described above, may made on a double needle bar warp knitting machine. The number and position of guide bars, the guide bar threading and gating and the guide bar movement, number and position of the needle beds, and stitch pattern may all be manipulated to create different results that may impact density, weight, strength, recoverability and elongation. The knit article 80 is created by a knitting method as shown in the diagram of FIG. 3A. The article 80 is created by first concurrently knitting first and second single parallel layers 40, 42 respectively. Then, the parallel layers 40, 42 converge and are knit together to form one uniform layer 43.

The first step of forming the first and second parallel layers 40, 42 is accomplished by threading or guiding a first yarn 44 into a pair of front guide bars 46, 48 which interact with a first needle bed 50 to form the first single layer 40. The second single parallel layer 42 is formed by threading or guiding a second yarn 52 into a pair of back guide bars 54, 56 which interact with a second needle bed 58 to form the second single layer 42 in parallel with the first layer 40.

The uniform layer 43 is created by altering the position of the guide bars. In particular, one of the back guide bars 54 is programmed to move to the front position and one of the front guide bars 46 is programmed to move to the back position. In this way, the first 44 and second 52 yarns are intertwined to form the uniform layer 43 which is made up of both first and second yarns.

It is appreciated that by alternating where the front and back guide bars knit (on the front needle bed or on the back needle bed) a pocket can be created if the sides are closed. In addition, by altering the density within the different layers, the uniform layer 43 and the parallel layers 40, 42 different layers may each have different characteristics and weight. Some of this may be done in fabrication.

The knitting process described above may continue to alternate between the single uniform layer 43 and two parallel layers 42, 44 to create a knit sheet 60. Alternatively, the process may only include the single step of either converging from two parallel layers 40, 42 to a single uniform layer 43, or diverging from the uniform layer and forming two parallel layers. Once the knitting has completed, the sheet 60 is removed from the knitting machine and cut into individual pieces.

As discussed above, the uniform layer 43 and parallel layers 40, 42 are cut to create a knit article of manufacture such as the article 10 described in detail above. As discussed previously, the first 12, second 14 and third 16, fourth 15, and fifth 17 segments of the first embodiment each have substantially the same weight. The stitch pattern enables more or less yarn to be used at any given time. This can impact the overall weight and density of the layer. During the formation of the uniform layer 43, the stitch pattern is altered from the stitch patterns used on the parallel layers 40, 42 so as to create a layer that closely resembles the weight of either one of the parallel layers 40, 42. In addition, it should be noted that the stitch pattern, and other variables may differ between the remaining layers 40, 42.

In addition, the courses per inch may be altered in a further effort to effect weight. In the uniform layer 43, the courses per inch were decreased relative to either of the parallel layers 40, 42 to further reduce the weight of the uniform layer.

Once the knit sheet 60 is complete, the sheet is removed from the knitting machine and cut to shape. Once cut, each piece is heat set. Prior to heat setting, the uniform layer 43, is stretched in the both the longitudinal and lateral directions so as to increase its width and length and thus further minimize the wales per inch and courses per inch. The courses and wales per inch may also be manipulated by the stitch pattern used as described above.

An alternative method for forming the article 10 involves a change in the manner in which the uniform layer is formed. As shown in FIG. 3B, the method involves the knitting, as described above, of two parallel layers 40, 42 using a front 50 and back 58 needle beds and front 46, 48 and back 54, 56 guide bars as described above. To create the parallel layers 40, 42, the first yarn 44 threaded in the front guide bars 46, 48 does not interact with the second yarn 52 threaded into the back guide bars 54, 56. Thus there is no interlacing of the first 44 and second 52 yarns. This results in the two, separate, parallel layers 40, 42.

The second part of this alternate method involves the creation of a second pair of parallel layers. The uniform layer 62 in this alternative method involves creating an alternative set of parallel layers 66, 68 of knit fabric. This is accomplished by causing one of the back guide bars to interact with the forward guide bars and needle bed to so that the first alternative parallel layer 66 now has three guide bars interacting with the forward needle bed. Finally, the second alternative parallel layer 68 has a single back guide bar interacting with the back needle bed. This results in an alternative first layer 66 with an increased weight and density of about 50% of either of the first parallel layers 40, 42. In addition, the alternative second parallel layer is 50% less in weight and density than either of the first parallel layers 40, 42.

To create the final product, the knitted sheet is removed from the machine and cut to shape. In addition, the second alternative parallel layer 68 is severed from the piece so that only three layers remain.

The above described method involved knitting a plurality of parallel layers and then a single uniform layer so that as the knit article is formed, the direction of manufacture only has one process occurring at a time, as shown in FIGS. 3A and 3B. In other words, as the knit article is being created on the knitting machine, the machine alternates between forming parallel layers and a uniform layer and those formation processes do not occur at the same time.

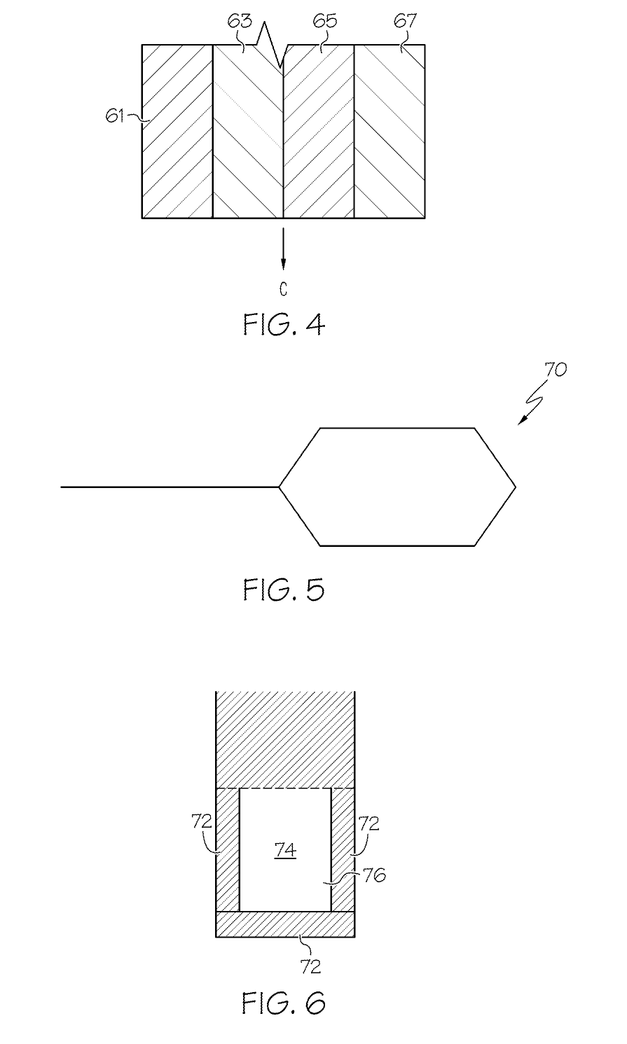

It is also contemplated that a knit article as described may be formed by knitting two or more parallel layers concurrently with knitting the single uniform layer. FIG. 4 provides a diagrammatic representation of this alternative process. As an example, multilayers may be knitted as described above in sections 61 and 65, and single uniform layers may be formed as described above in sections 63 and 67. The direction of manufacture of the knit article off the machine is indicated by arrow C.

It is also contemplated that because of the variety of multilayer and single layer combinations that may be produced using this method, a variety of combinations and permutations of resulting articles may be created. For example, an article having a singular uniform section may diverge into two parallel sections that are subsequently joined to form a loop 70. This design may be appropriate for use in tendon, ligament and joint repairs. One such example is shown in FIG. 5. Another alternative design may include the loop section described above, but also include side and bottom seams 72 in the form of uniform single layers so as to form an enclosed portion of the article, as shown in FIG. 6. Where the outer walls are formed by a uniform single layer and the pocket portion 74 is formed from spaced apart parallel layers 76.

A further alternative manner of making the article 10 described above, is by weaving. The methodology essentially employs the same principle in that a woven article 90 is formed by either converging two parallel woven layers into a single uniform layer, or by diverging a single uniform layer into two parallel woven layers. In either respect, the loom is prepared so that the ends simultaneously weave two separate layers in parallel. In other words, some of the ends weave a top layer and some of the ends weave a bottom layer where the top and bottom layers are completely independent of one another. At a predetermined point, at least some of the ends switch so that some of the top ends now weave on the bottom and vice versa. This results in an intertwined weave where the top and bottom layers are now interwoven to form a single conjoined layer made of yarn previously used to form the top and bottom layers. The weave pattern and picks per inch of the conjoined layer may be altered so that the density of the conjoined layer is substantially the same as that of either the parallel layers. In addition, ends may be dropped in the conjoined layer to further enable the conjoined layer to have a density substantially similar to either of the parallel layers.

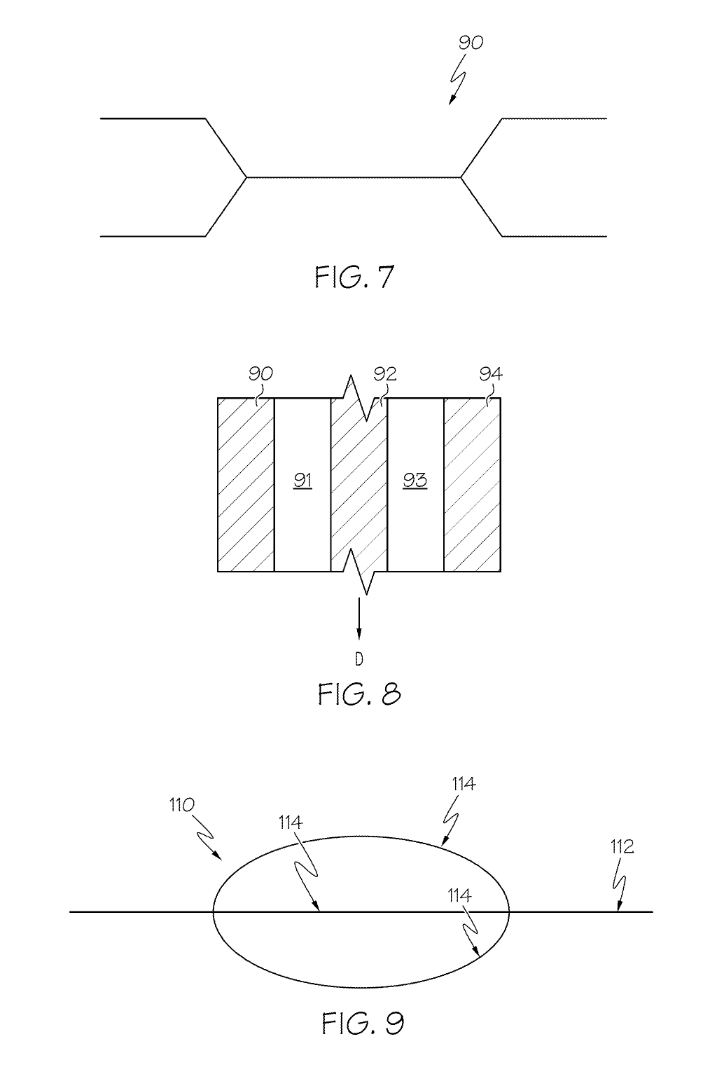

This weave pattern may continue or the ends may switch again to form separate parallel layers. It should be understood that the conjoined and parallel layers may be formed uniformly across the loom as shown diagrammatically in FIG. 7. However, the multilayered weave may also be created by forming conjoined and parallel layers at the same time across the loom as shown diagrammatically in FIG. 8. FIG. 8 shows multilayers represented by hashed areas 90, 92, 94 and uniform interwoven layers 91, 93 formed adjacent to each other and parallel to the direction of weaving as represented by arrow D. In addition, it is anticipated that combinations and permutations of these described arrangements may be realized to create a variety of end products. For example, another embodiment 110 using a woven method as described above may include a conjoined single layer 112, diverging into three parallel layers 114 and subsequently converging back to a single layer, as shown in FIG. 9.

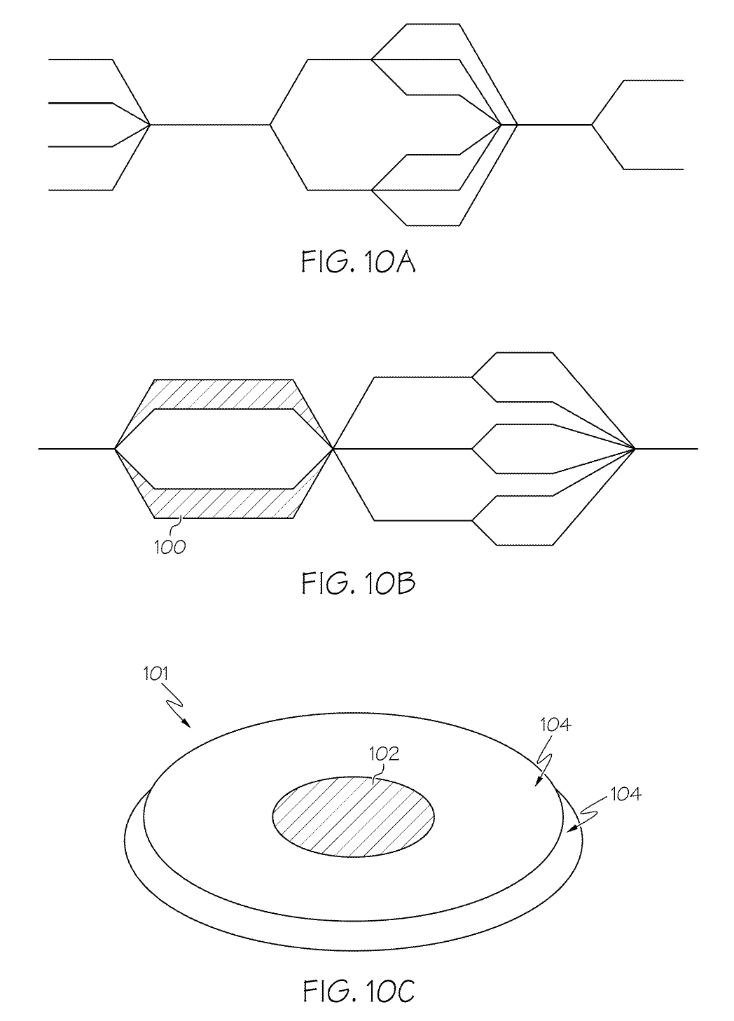

While the methodology and articles of such methodology have been described primarily as having two parallel layers converging to form a single uniform layer, it is contemplated that additional parallel layers may be added. For example, it is contemplated that a pair of parallel layers may converge to form a single uniform layer as described above, and then subsequently diverge to form three or more layers, with the pattern repeating as dictated by the design. Different combinations and permutations of the embodiments described herein are set forth in FIG. 10 by way of example and not in any way intended to limit the scope and purpose of the embodiments and methods described herein.

It is also anticipated that other materials and fibers 100 may be incorporated into the layers and designs to further create additional design features and attributes found attractive in the medical arts. For example, it may be advantageous to form a pair of parallel knit or woven layers having a non-woven material, drug eluting compound, or loose fibers sandwiched therebetween, as shown in FIG. 10B.

In addition, while the embodiments have been described and illustrated as being primarily linear, it is anticipated that an article made in accordance with the described methodologies may be circular, oval or other non-linear shape. In particular, it is anticipated that a circular or oval shaped embodiment 101 having a central uniform layer 102 and diverging layers 104 extending outwardly from the uniform layer may be made using the methods described. This embodiment may be particularly applicable to a hernia repair or the like.

In addition, while the embodiments described herein include articles made from biocompatible fibers, filaments and yarns, it is appreciated that this includes bioabsorbable and non-bioabsorbable. In particular, the embodiments and methods described herein may be particularly suited to include both. For example, it is anticipated that an article may include a uniform layer made of both bioabsorbable and non-bioabsorbable yarns the parallel layers may include at least one layer made of only bioabsorbable yarn and at least one made of only non-bioabsorbable yarn. Alternatively, a layer made of nonresorbable yarn may be sandwiched between two layers made of resorbable yarns.

It is also appreciated that the weight and density of each section may be significantly different. It is noted that while substantially the same means no more or less than 50%, it is anticipated that the methods and articles made by the methods and articles described herein may be adopted to create a pair of sections having first and second densities and then a third conjoined section having a density that is substantially greater than either the first or second section. In other words, it is anticipated that the method described herein could be adapted to create a third section density that is more than 2.5 times greater than the density of either the first or second sections. This is accomplished by altering the stitch or weave pattern, courses per inch, wales per inch, ends per inch, picks per inch, and the like, and any combination or permutation of these factors, so as to increase the density of the third conjoined section to at least 2.5 times the density of either the first or second section. Such a change may be advantageous in creating articles that require added strength but need to be smaller in size, such as a tie or fastener. If the section is, for example, three (3) times the weight of the other sections, it may be designed to be one-third the size. Such a design may be advantageous when using minimally invasive surgical techniques where the article may be delivered via a catheter. A smaller sized article used as an implant may cause less discomfort to a patient or decrease the recovery time.

In addition, while the embodiments described herein include articles made from biocompatible fibers, filaments and yarns, it is appreciated that this includes bioresorbable and non-bioresorbable materials. In particular, the embodiments and methods described herein may be particularly suited to include both. For example, it is anticipated that an article may include a uniform layer made of both bioresorbable and non-bioresorbable yarns, and the parallel layers may be made exclusively of either bioresorbable or non-bioresorbable yarns. Alternatively a layer of non-bioresorbable yarns may be sandwiched between two or more layers of bioresorbable yarns.

Even though numerous characteristics and advantages of the embodiments and methods described in the foregoing description and figures, these descriptions are illustrative only. Changes may be made in detail, especially in matters of size, shape, and orientation of parts within the principles of the invention to the full extent indicated by the broad general meaning of the terms in which the appended claims are attached.

We claim:

1. An article for medical use comprising:
a first segment having a first density;
a second segment having a second density;
and a third segment having a third density, whereby the first, second and third segments converge, and whereby the first, second and third densities are substantially the same.

2. The article of claim 1 wherein the first segment is formed having a first pattern, the second segment is formed having a second pattern and the third segment is formed having a third pattern.

3. The article of claim 1 wherein the first, second and third segments are knitted.

4. The article of claim 1 wherein the first, second and third segments are woven.

5. A method of forming a textile for medical use comprising the steps of:
forming a first segment having a first density;
simultaneously forming a second segment having a second density;
forming a third segment having a third density; and
simultaneously forming a fourth segment having a fourth density, whereby the first, second, third and fourth sements converge at one or more locations, and whereby the first, second, and third densities are substantially the same.

6. The method of claim 5 further comprising the step of severing the fourth segment from the article.

7. The method of claim 5 wherein the steps of forming the first, second, third and fourth segments are accomplished by knitting.

8. The method of claim 5 wherein the steps of forming the first, second, third and fourth segments are accomplished by weaving.

9. An article for implant made in accordance with claim 5.

10. A textile article for use in minimally invasive procedures, the article comprising:
a first segment; and
a second segment, the second segment having a first surface and a second opposing surface; and
a third segment, the third segment having a first surface and a second opposing surface, whereby indicia are located on the second and third segments to provide relative positioning indicators.

11. The article of claim 10 wherein the first surfaces of the second and third segments are proximate and the indicia are located on the second surfaces of each of the second and third segments.

12. The article of claim 11 wherein indicia are located on the first surfaces of each of the second and third segments.

13. The article of claim 11 wherein indicia comprise a longitudinal demarcation to the right of center.

14. The article of claim 12 wherein indicia comprise a longitudinal demarcation to the left of center on the first surface of each of the second and third segments.

15. The article of claim 10 further comprising incremental indicia to aid in measurement.

16. The article of claim 10 wherein the first, second and third segments converge.

17. The article of claim 10 wherein the weight of each of the first, second and third segments is substantially the same.

18. A method of making an article for medical use, comprising the steps of:
forming a first segment;
forming a second segment having a first surface and a second opposing surface;
forming a third segment having a first surface and a second opposing surface; and
providing relative position indicia on the second and third segments.

19. The method of claim 18 wherein the first surfaces of the second and third segments are proximate, and the step of providing relative position indicia comprises providing indicia on the first surfaces of the second and third segments.

20. The method of claim 18 wherein the first surfaces of the second and third segments are proximate, and the step of providing relative position indicia comprises providing indicia on the second surfaces of the second and third segments.

21. The method of claim 20 wherein the step of providing relative position indicia comprises providing longitudinal indicia to the left of center on the second surfaces of the second and third segments.

22. A method of manufacturing an article for medical use comprising the steps of:
threading a first yarn into a first pair of guide bars and knitting a first layer of fabric;
threading a second yarn into a second pair of guide bars and knitting a second layer of fabric concurrently with the first layer of fabric; and
causing at least one guide bar from the first pair and at least one guide bar from the second pair to change position so as to knit a third layer of fabric comprised of the first and second yarns, whereby the weight of the first, second and third layers are each substantially the same.

22. A method of manufacturing a medical textile comprising the steps of:
weaving a first yarn to form a first layer of fabric, the first layer of fabric having first ends;
concurrently weaving a second yarn to form a second layer of fabric, the second layer of fabric having second ends; and
causing at least some of the first ends and at least some of the second ends to change position so as to weave a third layer of fabric comprised of the first and second yarns, whereby the weight of the first, second and third layers are each substantially the same.

23. The method of claim 22 further comprising the step of causing the first and second ends to diverge so as to weave a fourth layer of first yarn and a fifth layer of second yarn.

24. The method of claim 22 wherein the first layer has a first number of picks per inch, the second layer has a second number of picks per inch and the third layer has a third number of picks per inch, wherein the first and second number picks per inch are greater than the third number of picks per inch.

25. The method of claim 24 wherein the first and second number of picks per inch are the same.

26. A textile for medical use comprising:
a first segment made from at least a first yarn and having a first density;
a second segment made from a least a second yarn, and having a second density;
and a third segment made from the intertwining of at least the first and second yarns and having a third density, and whereby the first, second and third densities are substantially the same.

27. The textile of claim 26 wherein the third segment is non-linear in shape.

28. An article for medical use comprising:
a first segment having a first density made of at least a first yarn;
a second segment having a second density made of at least a second yarn; and
a third conjoined segment to the first and second segments, the third conjoined segment having a third density made of the first and second yarns, whereby the third density is at least 2.5 times greater than either the first or second segment densities.
